(19) United States
(12) Patent Application Publication
(10) Pub. No.: US 2022/0257869 A1
(43) Pub. Date: Aug. 18, 2022

(54) Automated Seldinger Technique Device

(71) Applicant: Juan Arturo Siordia, Nogales, AZ (US)

(72) Inventor: Juan Arturo Siordia, Nogales, AZ (US)

(21) Appl. No.: 17/174,353

(22) Filed: Feb. 12, 2021

(51) Int. Cl. A61M 5/315; A61M 25/09

(52) U.S. Cl. CPC A61M 5/31566; A61M 2205/8206; A61M 25/09041

(57) ABSTRACT

A user-operated apparatus comprising of a body which holds a Raulerson syringe and contains a trigger mechanism to pull the plunger end of the Raulerson syringe to aspirate contents, all of which can be held and controlled with a single hand, and a mechanism housing a controller board, electrical circuit, and gears that allows for loading the guide wire into the Raulerson syringe.

### BACKGROUND OF THE INVENTION

### 1. Field of the Invention

The present invention is in the field of healthcare, particularly with procedures that include the Seldinger technique.

### 2. Discussion of the State of the Art

The Seldinger technique is a method developed to introduce guide wires and catheters into the body of patients with accuracy and care. It is commonly used in patients in the intensive care unit, operating room, or catheter suite. The technique involves introduction of a needle into the desired body location, loading a guide wire into the needle once the desired body fluid is expressed (or aspirated with a syringe), always maintaining wire control, removing the needle, introducing a tube or catheter onto the wire, and then removing the guide wire afterwards. Ultrasonography while performing the procedure is common to improve accuracy and reducing peri-procedural complications. However, one must release the ultrasound probe from one hand to load the guide wire into the needle since it is a two-handed process. Releasing the ultrasound probe not only loses real-time visualization of the needle location, but it may also move the body tissue and needle, leading to loss of the desired needle location when trying to load the guide wire.

Therefore, what is clearly needed is a device that allows for continuous ultrasound visualization during insertion of the wire while maintaining the needle steady.

### BRIEF SUMMARY OF THE INVENTION

In one embodiment of the invention, an apparatus is provided, comprising a body which holds a Raulerson syringe and contains a trigger mechanism to pull the plunger end of the Raulerson syringe to aspirate contents, all of which can be held and controlled with a single hand, and a mechanism housing a controller board, electrical circuit, and gears that allows for loading the guide wire into the Raulerson syringe.

Also in one embodiment, a switch is used to activate the guide wire loading mechanism. Also in one embodiment, the mechanism can be turned on using a circuit which includes an on-and-off switch to initiate the device. Also in one embodiment, a geared mechanism is activated to push a guide wire into the plunger of the Raulerson syringe and further into the desired blood vessel or any other location within a body of the patient. Also in one embodiment, the geared mechanism to push the guide wire is comprised of a servo-based mechanism. Also in one embodiment, the electrical circuit powered by a battery. Also in one embodiment, the trigger mechanism comprises of a system that holds plunger end of the Raulerson syringe and can be moved by a squeeze of a single hand while the body of the Raulerson syringe is stabilized by the body of the apparatus.

In another aspect of the invention, a method is presented comprising of loading a Raulerson syringe and guide wire into the apparatus, allowing the user to free the other hand to control an ultrasound probe while introducing the needle into the body of a patient by use of the apparatus, once the desired aspirate is contained in the Raulerson syringe, the operator activates the geared mechanism via a switch to load a guide wire into the Raulerson syringe and eventually into the desired location of the body, allowing the user to continuously monitor the introduction of the guide wire into the desired location of the patient via ultrasound, turning off the mechanism via a switch once the desired length of the guide wire is introduced into the body, and removing the Raulerson syringe with the needle from the apparatus while holding the guide wire in place.

### BRIEF DESCRIPTION OF THE SEVERAL VIEWS OF THE DRAWINGS

FIGS. 1A, 1B, 1C, 1D, and 1E illustrate a left-sided, a right-sided, a top, a rear, and an isometric view, respectively, according to an embodiment of the present invention.

FIG. 2 presents circuitry and contents contained within a handle of an embodiment of the present invention.

FIG. 3A-3D illustrate processes for preparing an embodiment of the present invention.

FIG. 4 presents an embodiment of the present invention in use, particularly during introduction of the needle into a body.

FIG. 5 illustrates a top view of an embodiment of the present invention, presenting the contents within the gear box and the motion of the gears once activated.

FIG. 6 presents an example of a guide wire being introduced into the body with activation of an embodiment of the present invention.

FIG. 7 is a process flow chart illustrating steps for preparing and using the device of FIG. 1.

It will be appreciated that for purposes of clarity and where deemed appropriate, reference numerals have been repeated in the figures to indicate corresponding features, and that the various elements in the drawings have not necessarily been drawn to scale to better show the features.

### DETAILED DESCRIPTION OF THE INVENTION

The inventor provides an automated single hand-held device for inserting the guide wire into a selected body location. The present invention is described in enabling detail in the following examples, which may represent more than one embodiment of the present invention.

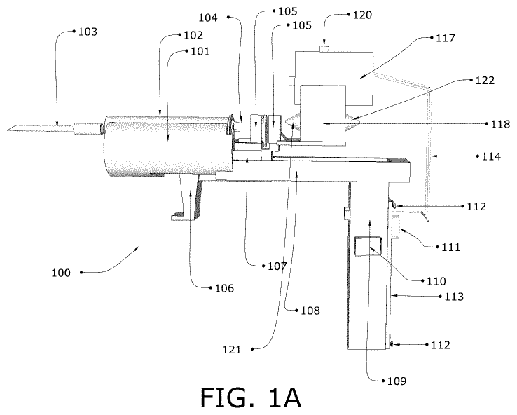

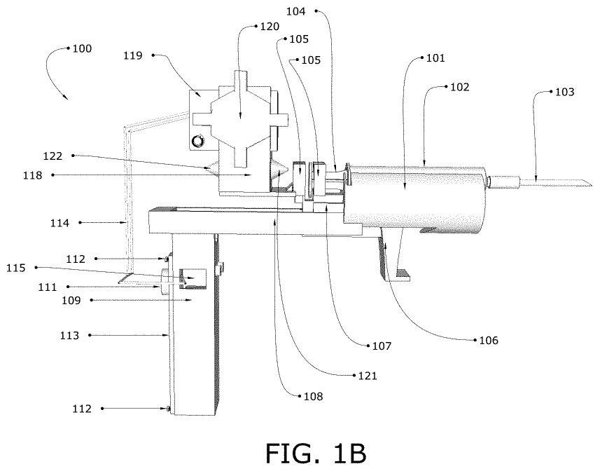

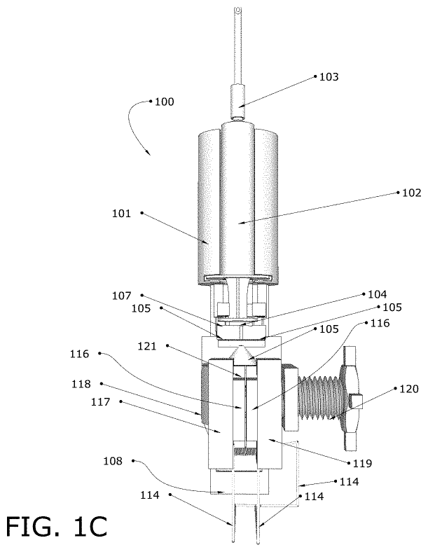

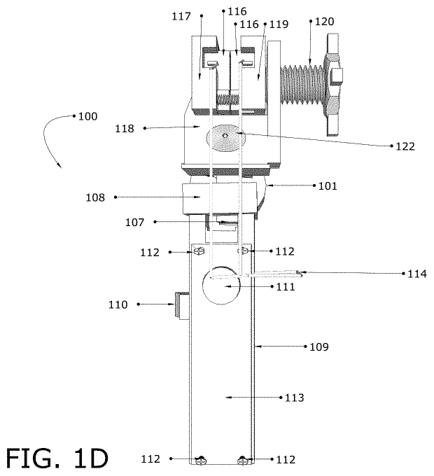

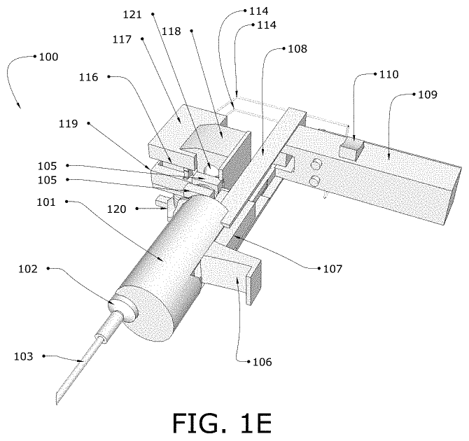

FIG. 1 presents views of one embodiment of the automated Seldinger technique device 100. FIG. 1A presents a left-sided view of one embodiment of the automated Seldinger technique device 100. The body of the device 101 contains a compartment where the operator can insert a Raulerson syringe 102. The syringe 102 is inserted with a needle 103 attached to the tip. The plunger of the syringe 104 is inserted into a plunger holder 105 of the device which is attached to the trigger handle 106 via the trigger body 107. The trigger housing unit 108 is attached to the body of the device 101 and houses the trigger mechanism (including the plunger holder 105, trigger body 107, and trigger handle 106) so as to allow it to move forwards and backwards whenever the trigger handle 106 is pulled towards or pushed away from the device handle 109. The device handle 109 also serves as a hollow compartment to house the circuitry and switches that power the device. Switches include the on-and-of switch 110 and a push button 111. Screws 112 are used to attach the circuitry compartment cover 113 to the device handle 109. Wires 114 connected to the controller board and circuitry housed within the device handle 109 go through a small side window 115 of the handle 109 and connect to the servo units 116 contained within servo holders of the device. Servo holders include a fixated servo holder 117 to the gear box 118 and a loose servo holder 119. The loose servo holder 119 is pushed towards the fixated servo holder 117 via turning of a large screw 120. This allows the gears connected to the servo units to come into contact within the gear box 118. The gear box 118 also includes two cone-shaped exit points—one in the front 121 and another in the rear 122—to allow for passage of a guide wire through the gear box 118, the plunger holder 105, and into the plunger 104 of the Raulerson syringe 102. FIGS. 1B, 1C, 1D, and 1E present a right-sided, a top, a rear, and an isometric view of one embodiment of the automated Seldinger technique device 100, respectively.

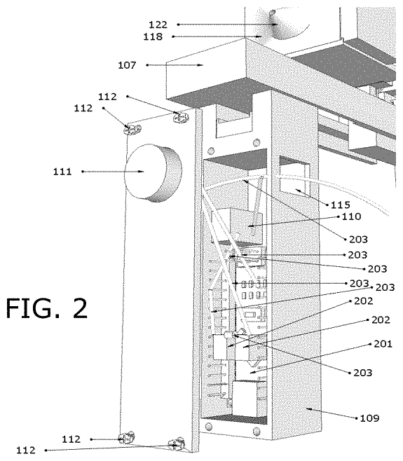

FIG. 2 presents a view of the circuitry housed within the device handle 109. By removing the screws 112, the circuitry compartment cover 113 can be removed to reveal the circuitry inside. Contents include a controller board 201 that is powered by batteries 202 that are regulated by the on-and-off switch 110. Turning the on-and-off switch 110 to “on” will power the controller board 201. Connections are created with wires 203. The push button 111 also controls the power from the batteries 202 to the servo units 116 seen in FIG. 1. Holding down the push button 111 turns on the servo units 116; releasing the push button 111 turns off the servo units 116.

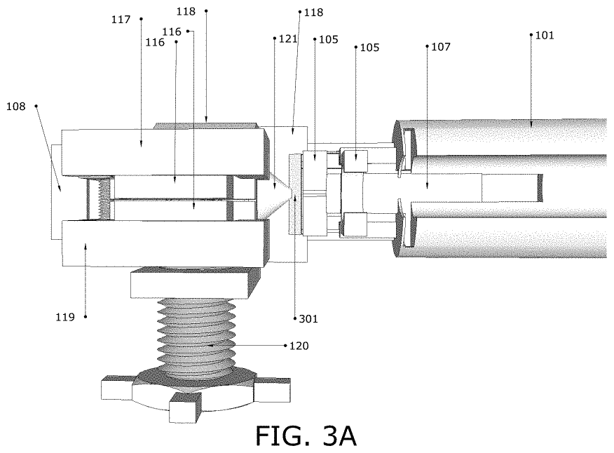

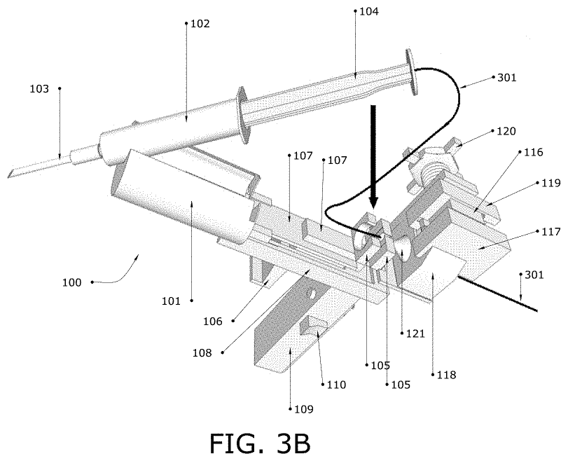

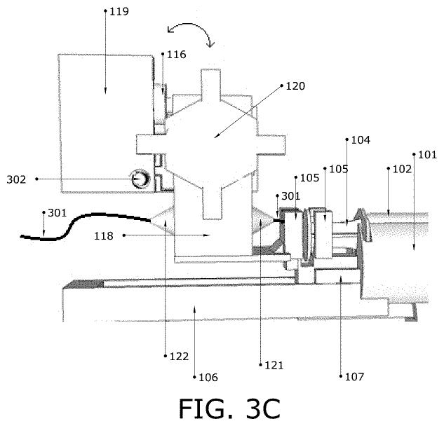

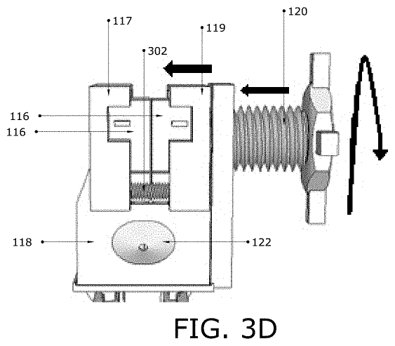

FIG. 3 illustrates the process of preparing the automated Seldinger technique device 100. FIG. 3A presents the guide wire 301 being introduced into the front cone-shaped exit point 121 to enter the gear box 118 and exit from the rear cone-shaped exit point 122. FIG. 3B shows the Raulerson syringe 102 being loaded onto the device. The guide wire 301 is loaded into the Raulerson syringe 102 through the plunger 104. The Raulerson syringe 102 itself is snapped onto the body of the device 101, and the plunger 104 is snapped into the plunger holder 105. When the syringe is in place, the guide wire 301 can be pulled further back to straighten it. While loading the guide wire 301 through the gear box 118 should be as simple as pushing it into the front cone-shaped exit point 121 until it exits out of the rear cone-shaped exit point 122, there may be times where the guide wire may not go easily through. For if this issue occurs, FIG. 3C shows the ability of the loose servo holder 119 with its respective servo unit 116 pivoting on a shaft 302 connected to the fixated servo holder 117. Lifting the loose servo holder 119 backwards (as displayed in FIG. 3C) will reveal the inside of the gear box 118. This allows the operator to visualize the guide wire 301 go through the gear box 118 fully and realign it if necessary. After the guide wire 301 is fully through the gear box 118, the loose servo holder 119 can be rotated back into the gear box 118. The loose servo holder 119 is secured in place by turning the large screw 120 clockwise, as demonstrated in FIG. 3D. This also ensures that the two servo units 116 are abutting each other.

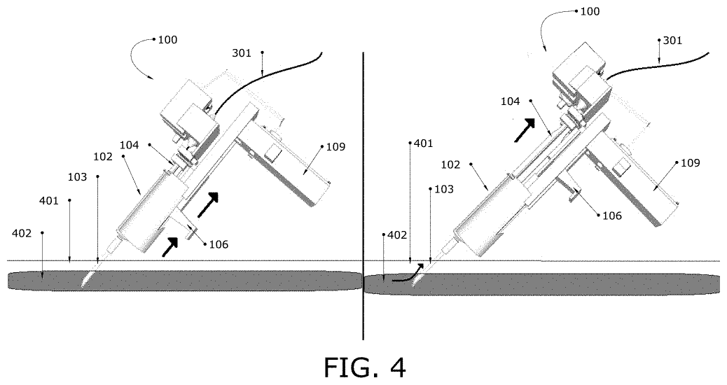

FIG. 4 presents an example of the automated Seldinger technique device 100 once it is prepared for use. The needle 103 is introduced into the skin 401 of the patient while the trigger handle 106 pulled back toward the device handle 109 to induce a sucking force within the Raulerson syringe 102. Once the needle 103 reaches the desired body location—such as a blood vessel 402 as demonstrated in the figure—the trigger mechanism will pull the plunger of the syringe 104 to allow aspiration of body fluid contents through the needle 103 and into the syringe 102. With the syringe being transparent, the operator will be able to see the aspirated contents. The process presented in FIG. 4 can be performed with a single hand, allowing the other hand to hold an ultrasound probe and maintain continuous ultrasound guidance if desired.

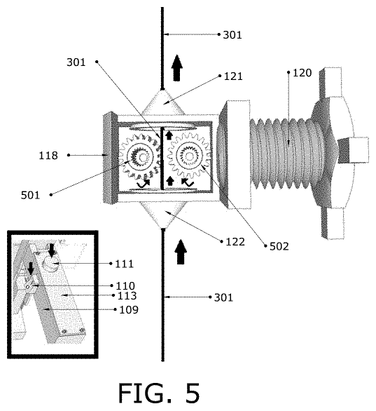

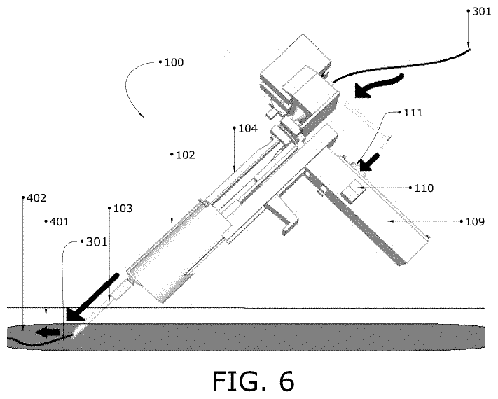

When the operator is confident that the needle is in the desired location, the guide wire 301 can be introduced into the targeted compartment. FIG. 5 presents a top view of the gear box 118 without the fixed 117 or loose 119 servo holders and their respective servo units 116. Two gears 501 502 are housed inside the gear box 118 and can be turned when their attached servo units 116 are activated. By flipping the on-and-off switch 110 to “on” and holding down the push button 111, the servo units 116 will be activated. The servo unit 116 contained in the fixed servo holder 117 will spin the respective gear 501 counter-clockwise. The servo unit 116 contained in the loose servo holder 119 will spin the respective gear 502 clockwise. This will push the guide wire 301 toward the front cone-shaped exit point 121 and into the plunger 104 of the Raulerson syringe 102. FIG. 6 illustrates the guide wire going through the automated Seldinger technique device 100 and advancing into the target location—such as a blood vessel 402 as demonstrated. Releasing the push button 111, will deactivate the servo units 116. Flipping the on-and-off switch 110 to “off” will also deactivate the servo units 116.

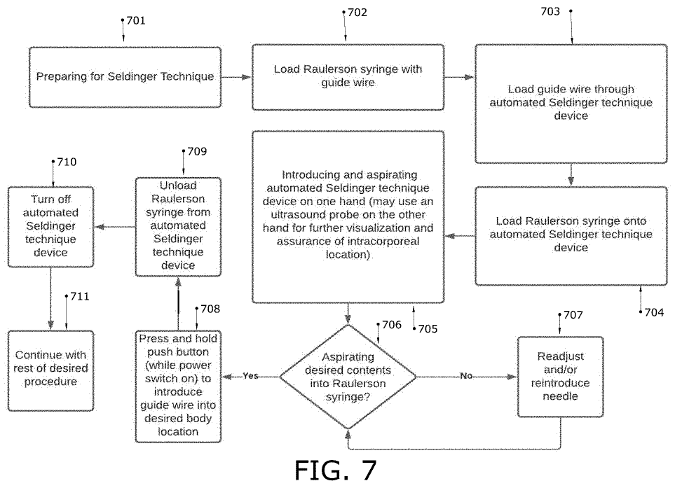

FIG. 7 provides a flowchart of a method for using the automated Seldinger technique device 100. In state 701, the operator prepares for the Seldinger technique required in a procedure. In state 702, the guide wire 301 is loaded into the Raulerson syringe 102. In state 703, the other end of the guide wire 301 is inserted through the front cone-shape exit point 121, through the gear box 118, and out through the rear cone-shape exit point 122. In stage 704, the Raulerson syringe 102 with the loaded guide wire 301 is inserted into the space provided by the body of the device 101 with the plunger of the syringe 104 being inserted into the plunger holder 105. In stage 705, the needle 103 attached to the Raulerson syringe is advanced into the skin of the patient 401 with the automated Seldinger technique device 101. Aspiration of the syringe 102 is performed by single-handedly squeezing the trigger handle 106 with one finger toward the device handle 109. An ultrasound probe can be used with the other hand to continuously visualize the needle advancement. In state 706, the syringe 102 is surveyed for the desired bodily fluid. If, in state 706, the desired contents are not aspirated, then in state 707, the angle of the needle is readjusted or reintroduced into the body. Aspirating the desired contents is attempted again. If, in state 706, the desired bodily fluid is aspirated, then in state 708, the operator activates the device 100 by turning the on-and-off switch 110 to “on”. The operator then holds the push button 111 down to advance the guide wire 301 through the syringe 102 and into the desired location within the body. Note that states 706, 707, and 708 can be performed with continuous ultrasound surveillance with an ultrasound probe in the other hand. After the guide wire 301 is advanced sufficiently, then in state 709, the syringe 102 is removed from the automated Seldinger technique device 100 via the, opposite maneuvers performed in states 703 and 704. In state 710, the automated Seldinger technique device 100 is powered down by flipping the on-and-off switch 110 to “off”. In state 711, the operator can then continue with the rest of the procedure.

It will be apparent to one with the skill in the art that the automated line placement device may be provided using some or all of the mentioned features and components without departing form the spirit and scope of the present invention. It will also be apparent to the skilled artisan that the embodiments described above are specific examples of a single broader invention which may have greater scope than any of the singular descriptions taught. There may be many alternatives made in the descriptions without departing from the spirit and scope of the present invention.

1. An apparatus comprising:
a body which holds a Raulerson syringe and contains a trigger mechanism to pull the plunger end of the Raulerson syringe to aspirate contents, all of which can be held and controlled with a single hand;
a mechanism housing a controller board, electrical circuit, and gears that allows for loading the guide wire into the Raulerson syringe.

2. The apparatus of claim 1, wherein a switch is used to activate the guide wire loading mechanism.

3. The apparatus of claim 2, wherein the mechanism can be turned on using a circuit which includes an on-and-off switch to initiate the device.

4. The apparatus of claim 2, wherein a geared mechanism is activated to push a guide wire into the plunger of the Raulerson syringe and further into the desired blood vessel or any other location within a body of the patient.

5. The apparatus of claim 4, wherein the geared mechanism to push the guide wire is comprised of a servo-based mechanism.

6. The apparatus of claim 2, wherein the electrical circuit powered by a battery.

7. The apparatus of claim 1, wherein the trigger mechanism comprises of a system that holds plunger end of the Raulerson syringe and can be moved by a squeeze of a single hand while the body of the Raulerson syringe is stabilized by the body of the apparatus.

8. A method comprising:
Loading a Raulerson syringe and guide wire into the apparatus (such as mentioned in claim 1);
Allowing the user to free the other hand to control an ultrasound probe while introducing the needle into the body of a patient by use of the apparatus (such as mentioned in claim 1);
Once the desired aspirate is contained in the Raulerson syringe, the operator activates the geared mechanism (such as mentioned in claim 4) via a switch (such as mentioned in claim 2) to load a guide wire into the Raulerson syringe and eventually into the desired location of the body;
Allowing the user to continuously monitor the introduction of the guide wire into the desired location of the patient via ultrasound;
Turning off the mechanism via a switch (such as mentioned in claim 2) once the desired length of the guide wire is introduced into the body;
Removing the Raulerson syringe with the needle from the apparatus (such as mentioned in claim 1) while holding the guide wire in place.
